(19) United States
(12) Patent Application Publication
(10) Pub. No.: US 2007/0050046 A1
(43) Pub. Date: Mar. 1, 2007

(54) Methods for generating a signal indicative of an intended movement

(76) Inventor: Apostolos P. Georgopoulos, Minneapolis, MN (US)

Correspondence Address: MANDEL & ADRIANO, 55 SOUTH LAKE AVENUE, SUITE 710, PASADENA, CA, 91101, US

(21) Appl. No.: 11/492,732

(22) Filed: Jul. 25, 2006

Related U.S. Application Data
(60) Provisional application No. 60/702,262, filed on Jul. 25, 2005.
(60) Provisional application No. 60/702,188, filed on Jul. 25, 2005.

(51) Int. Cl. A61F 2/48; A61B 5/04; G09B 21/00

(52) U.S. Cl. 623/24; 340/825.19; 600/544

(57) ABSTRACT

The invention provides methods for generating signals indicative of intended movements which comprises measuring EEG signs from the brain or nervous system of a moving subject; processing the EEG signals to generate a mathematical model of the intended movement that correlates EEG signals with the intended movement; and obtaining the signal indicative of the intended movement.

This application claims the benefit of the filing dates of U.S. Ser. No. 60/702,188, filed Jul. 25, 2005 and U.S. Ser. No. 60/702,262, filed Jul. 25, 2005, the contents of both of which are incorporated by reference into the present application.

This invention was made with Government support through the Minneapolis VA Medical Center and under Grant awarded by the Department of Energy to the MIND Institute (Albuquerque, N.Mex.). The Government has certain rights in this invention.

Throughout this application, various publications are referenced within parentheses. The disclosures of these publications are hereby incorporated by reference herein in their entireties.

### FIELD OF THE INVENTION

The present invention relates to methods for processing EEG signals indicative of an intended movement to enable communication and control of devices, including motor prostheses.

### BACKGROUND OF THE INVENTION

In the past several years, considerable progress has been made in the precision of movements controlled remotely through the use of microelectrode arrays implanted in a subject's nervous system (Wolpaw et al. 2000; Taylor et al. 2002; Wessberg and Nicolelis 2004). Improvements in hardware and data processing, in conjunction with closed-loop feedback training, have shown good promise for providing functional movement to the neurologically impaired. At the same time, substantial improvements have been achieved in signal processing, training techniques and interpretation of data in noninvasive brain-machine interface (BMI) using electroencephalography (EEG) (McFarland et al. 1997; Wolpaw et al. 2000; Wessberg and Nicolelis 2004; Scherer et al. 2004; Hinterberger et al. 2004). For example, four human subjects (two of whom were paralyzed but had normal arm function), working with 64-lead EEG and adaptive algorithms, demonstrated two-dimensional (2-D) control over a computer cursor following many weeks of training sessions (Wolpaw and McFarland 2004).

However promising these advances may be, there are drawbacks to both approaches. For example, information transfer rates for both invasive and noninvasive techniques are often low (Wolpaw et al. 2000). Cortical electrode implantation carries inherent risks and may not be an available option for some patients. Furthermore, studies of implant longevity in rodents, while encouraging due to the limited fibrous encapsulation, have shown viability on the order of only months and not years (Vetter et al. 2004). On the other hand, EEG-based BMI has focused on keyboard interfaces controlled by effortful changes in cortical rhythm(s), thus requiring substantial training time and providing limited variables over which a subject may gain control.

In this study, we explored the feasibility of using real-time electroencephalography (EEG) to predict movement trajectories, e.g., 2-D movement trajectories, in a drawing task. For this purpose, we applied analysis methods developed previously in the context of neurophysiological recordings (Georgopoulos et al. 1988).

Surprisingly, the results of the experiments described below demonstrate an excellent prediction of movement trajectory by real-time EEG.

### SUMMARY OF THE INVENTION

The invention provides methods for generating signals indicative of intended movements which comprises measuring EEG signs from the brain or nervous system of a subject, e.g., a moving subject; processing the EEG signals to generate a mathematical model of the intended movement that correlates EEG signals with the intended movement; and obtaining the signal indicative of the intended movement.

### BRIEF DESCRIPTION OF THE FIGURES

FIG. 1 shows actual (blue lines) and predicted (fuchsia lines) X- and Y-trajectories using 20 time-points per sensor (see text, Eqs. 3, 4), based on the whole sample. A Unsmoothed predictions, B cubic-spline smoothed predictions, C X-Y plots of data in B.

FIG. 2 shows actual (blue lines) and predicted (fuchsia lines) X- and Y-trajectories using 20 time-points per sensor for training and test data. Coefficients were calculated from the training set and were applied to predict the test set.

FIG. 3 shows cross validated prediction of trajectory based on EEG.

### DETAILED DESCRIPTION OF THE INVENTION

Definitions:

As used in this application, the following words or phrases have the meanings specified.

The term “processed EEG signal” used herein is defined to mean a representation of a neurophysiological measurement of the electrical activity of the brain acquired from a subject (e.g., a mammal such as a primate) undergoing an intended movement. The processed neural signal may be generated by: acquiring and/or measuring EEG signals from a subject; and processing the EEG signals so that the final processed neural signal is organized to represent the subject's intended movement. The processed EEG signal may be represented in the form of electric fields/currents.

The acquiring step comprises detecting the neural activity, using a sensor(s) that detects both the steady state EEG activity and the change over steady state EEG activity. The processing steps comprise signal processing steps that will organize the acquired EEG signal to represent the intended movement.

The term “intended movement” used herein is defined to mean a movement of a limb of a subject or a computer screen pointing device, to a desired target location. An intended movement can include an actual movement. The limb may be a natural arm or leg, or a prosthetic device that may be attached directly or indirectly to the body. The subject may move any part of the limb to the desired target location. For example, the subject may move a part of the arm, such as the finger, hand, forearm, elbow, or shoulder. The subject may move a part of the leg such as the toes, foot, ankle, heel, knee, or thigh. Alternatively, the intended movement may comprise moving a computer screen pointing device to any desired location on the computer screen.

The term “control signal” used herein is defined to mean the processed EEG signal described above that has been further processed by translating the target location into an instruction (e.g., an electronically coded instruction) that directs a desired action by the subject, such as reaching to the target location. The instruction may interface with the subject's limb or a prosthetic device (e.g., an electronic device) to permit the subject to perform an intended movement.

The term “direct a desired action by a subject” used herein is defined to mean an intended movement, by a subject, of a natural limb or a device to a target location. The control signal described above instructs the subject's natural arm or the device to move to the intended target location. The subject may intend to move/manipulate the natural limb (e.g., the subject intends to move any part of the arm or leg), a prosthetic limb that may be attached or not attached to the subject, or manipulate a computer screen pointing device that is not attached to the subject.

In order that the invention herein described may be more fully understood, the following description is set forth.

Methods for Generating EEG Signals

The present invention provides methods for generating a signal indicative of an intended movement of a subject.

In one embodiment, the method comprises measuring EEG signals from the subject, e.g., from the brain or nervous system of a moving subject. Typically, a sensor (e.g., an electrode) is attached to the subject, e.g., the subject's scalp, and the sensor detects the activity of large groups of neurons. Single or multiple sensors may be used. For example, at least about 20 electrodes to about 64 electrodes can be used. In another example, at least about 5 electrodes to about 100 electrodes can be used.

The method further comprises processing the EEG signals to generate a mathematical model of the intended movement that correlates EEG signals with the intended movement and obtaining a signal indicative of the intended movement.

In another embodiment, the method may further comprise processing the measured EEG signals together with concurrently measured coordinates of the subject's actual movements to generate a mathematical model that, given new EEG signals, can generate a signal indicative of a future intended movement by the subject.

In accordance with the invention, the EEG signal can be processed by performing a multivariate linear regression. For example, the multivariate linear regression involves obtaining geometric coordinates of the movement of the subject in space (e.g., designated X and Y (two dimensional space), and optionally Z (three dimensional space)) while simultaneously measuring EEG signals and using the geometric coordinates so obtained together with EEG signals so measured to arrive at a mathematical model of the intended movement. In multivariate linear regression, the geometric coordinates of the movement trajectory can be designated the dependent variables and the EEG signals as the independent variables. The regression analysis can establish X-, Y-, and optionally Z-coefficients (i.e., weighting factors) for each sensor (electrode), so that this regression analysis can establish X-, Y-, and optionally Z-coefficients (i.e., weighting factors) for each sensor (also referred to herein as a lead), and predicting movement trajectory by weighting ongoing values of the EEG signals by the corresponding X-, Y-, and optionally Z-coefficients, and summing linearly across leads. Merely by way of example, the X-, Y- and optionally Z-, coordinate data can be collected simultaneously.

In accordance with the practice of the invention, the method can further comprise the step of translating the signal so obtained so as to generate a control signal. The control signal can direct a desired action by the subject. For example, the desired action can include having the subject reach using their limb (e.g., an arm or a leg). The limb can be the subject's own or a prosthetic device attached to the subject. Alternatively, the signals so obtained can be used to enable communication and control of devices, including motor prostheses.

An EEG signal originates from the brain of a subject, for example a mammal such as a primate, while the subject is moving an appendage such as a limb. The methods described herein can generate processed neural signals in subjects, including but not limited to, a human, dog, or cat. To process the EEG signal and obtain a signal indicative of the intended movement, one can perform a multivariate linear regression.

An EEG signal can be generated while a movement is made. For example, using electrodes attached to the scalp, or placing them subdurally, electro-physiological techniques detect changes in electrical potential.

The EEG signal may be conditioned and transmitted for input to the acquisition system, e.g. smoothing of EEG signals, Fourier transforms, analog-to-digital conversion. Signal conditioning, if required, may include signal amplification and/or filtering, as appropriate. Signal conditioning may involve pre-processing and/or filtering the EEG signals to remove artifactual signals prior to being used to predict movement trajectory. Signal conditioning requirements depend on the type of sensor used and on the input specifications of the acquisition system. Signal conditioning is a standard step in the art of data acquisition systems. The acquisition system measures the sensor signal.

In one particular embodiment, the invention provides a method of predicting how a subject is going to move a device, e.g., joystick, in two-dimensional space, comprising (a) in an initial training period, establishing how the subject's brain EEG signals vary when the subject moves the device in two-dimensional space; (b) using the data to establish a mathematical model that correlates EEG signals with movement of the device in two-dimensional space; and (c) given a set of EEG signals by the subject, using this mathematical model to predict how the subject is going to move the device. In this embodiment, the EEG signals can be measured using sensors (e.g., leads) attached to the subject, e.g., on the subject's skull. About 26-64 sensors (leads) can be used to gather the data. The mathematical model may be established by measuring the geometric coordinates (X, Y, and optionally Z) of the movement of the subject in space while simultaneously measuring EEG signals and performing a multivariate linear regression using the geometric coordinates of the movement trajectory as the dependent variables and the EEG data from the sensors (leads) as independent variables; using this regression analysis to establish X-, Y-, and optionally Z-coefficients (i.e., weighting factors) for each sensor (lead); then predicting movement trajectory by weighting ongoing values of the EEG signals by the corresponding X-, Y-, and Z-coefficients; and summing linearly across the sensor (leads). The EEG signals and geometric coordinate data may be collected simultaneously.

The invention further provides an apparatus comprising a receiver to receive signals, the signals representing EEG signals; and an analyzer to analyze the signals to predict movement trajectory.

The following examples are presented to illustrate the present invention and to assist one of ordinary skill in making and using the same. The examples are not intended in any way to otherwise limit the scope of the invention.

### EXAMPLES

### Example 1

### Magnetoencephalographic Signals Predict Movement Trajectory in Space

Ten right-handed subjects (five women and five men) participated in these experiments as paid volunteers (age range, 23-41 years; mean±SD, 30±6 years). The study protocol was approved by the appropriate institutional review boards. Informed consent was obtained from all subjects prior to the study according to the Declaration of Helsinki. Stimuli were generated by a computer and presented to the subjects using an LCD projector.

Subjects were presented with a red fixation point surrounded by a pentagon. When the fixation point turned green, the subjects drew the shape continuously for 45 s by moving an X-Y joystick using their right hand. The joystick was located at arm's length and out of the visual field. Subjects were instructed to fixate on the central point throughout the task. They were also instructed to copy the shape counter-clockwise at their own speed. No visual feedback was provided. The fixation point and the pentagon were presented to the subjects using a periscopic mirror system which placed the image on a screen approximately 62 cm in front of the subject's eyes. The pentagon subtended approximately 10° of visual angle.

MEG data were collected using a 248 sensor whole-head axial-gradiometer system (Magnes 3600WH, 4D-Neuroimaging, San Diego, Calif.). MEG data, electrooculogram data, and joystick output (0.1-400 Hz) were acquired simultaneously at 1017.25 Hz. The cardiac artifact was removed using event synchronous subtraction (Leuthold 2003). X-Y joystick coordinates were determined by converting from mV to end-of-joystick excursion.

The first step in data analysis was a multivariate linear regression in which the time courses of the 248 sensors were the independent variables and the corresponding time courses of the X and Y coordinates of the joystick were the dependent variables. This analysis was implemented using the double-precision fast Givens transformation of the IMSL statistical and mathematical library, called from FORTRAN programs (Compaq Visual FORTRAN Professional edition version 6.6B). This analysis yielded X- and Y-coefficients for each sensor. Next, predicted X- and Y-trajectories were computed by a linear summation of the weighted time-varying contributions from the 248 sensors, as follows:

$$\begin{matrix}{X}_{t} = {a}_{x} + {{\sum }_{i}}^{248} {b}_{ix} {S}_{i} ( t ) & ( 1 ) \\ {Y}_{t} = {a}_{y} + {{\sum }_{i}}^{248} {b}_{iy} {S}_{i} ( t ) & ( 2 )\end{matrix}$$

where Xₜ, Yₜ, are the predicted X- and Y-trajectories at time t; aₓ, a_{y} are constants; bᵢₓ, b_{iy} are X- and Y-regression coefficients for sensor i; and Sᵢ (t) is the signal from sensor i at time t. In subsequent analyses the independent variables consisted of the above plus k=19 additional sample points of the 248 MEG signals preceding the currently predicted trajectory point at t, as follows:

$$\begin{matrix}{X}_{t}^{′} = {a}_{x}^{′} + {{\sum }_{i}}^{248} {{\sum }_{k}}^{k - 0 , 19} {b}_{ikx} {S}_{i} ( t - k ) & ( 3 ) \\ {Y}_{t}^{′} = {a}_{y}^{′} + {{\sum }_{i}}^{248} {{\sum }_{k}}^{k = 0 , 19} {b}_{iky} {S}_{i} ( t - k ) & ( 4 )\end{matrix}$$

This is a reasonable procedure, since changes in activity in most motor-related cortical areas precede movement onset. The quality of the prediction was quantified by calculating the Pearson correlation coefficient between the ms-by-ms actual and predicted data. Summary statistics (median, range) were obtained from pooled X- and Y-data across subjects. Finally, we carried out cross-validation analyses in which only the first half (22,500 time points; “training” set) were used for the calculation of weighting coefficients from the first half (22.5 s of the data; “training” set). Then trajectory predictions were computed (using Eqs. 3, 4) for both the training set as well as for the remaining cross-validated data points (“test” set). This cross-validation is important as a test of the feasibility of this approach to control a prosthetic device in real-time.

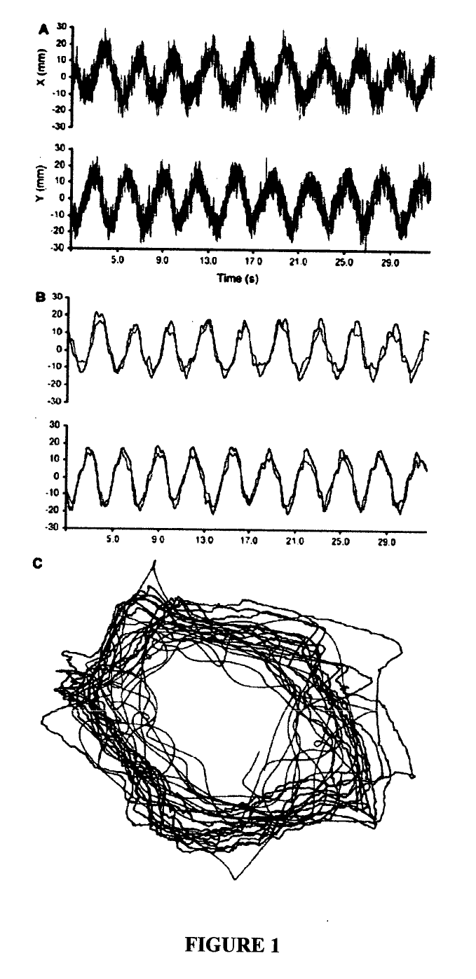

FIG. 1a shows an example from one subject of actual (blue lines) and predicted (fuchsia lines) X- and Y-trajectories, without smoothing, using Eqs. 3 and 4; very similar, good predictions were also obtained in the remaining subjects. The modulated neural prediction disappeared when the neural data were shuffled in time (data not shown), which means that the trajectory information resides in the temporal sequence of the MEG signal. The prediction was further improved following smoothing (FIG. 1b), and yielded 2-D trajectories practically indistinguishable from the actual movements (FIG. 1c). All predictions were of the same high quality for both X- and Y-data.

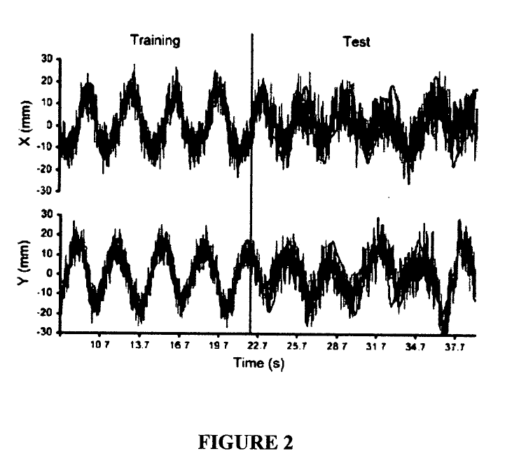

Next, we evaluated the robustness of these results by cross-validating the predictions between the first and second half of the data. As can be seen in FIG. 2, the predictions for the test set were very good, although a higher variance was present. Overall, these analyses documented the robustness of the results and the validity of the approach, as follows. The correlation coefficients between the actual and predicted trajectories in the first half were high (unsmoothed data median r=0.91, range 0.83-0.94; smoothed data: median r=0.97, range 0.95-0.99), and remained high in the second, cross-validated half (unsmoothed data: median r=0.76, range 0.59-0.86; smoothed data: median r=0.85, range 0.68-0.92).

These results show, for the first time, that there is adequate and robust information in the non-invasive MEG signal for real-time prediction of drawing movement trajectories. Moreover, this is the first time that such information of high quality has been extracted from single unaveraged trials. This should be very easy to implement in real time, given that the time-consuming part of the procedure is calculating the X-Y coefficients for each sensor, which can be done at leisure and for an extensive training set. In contrast, the calculation of the prediction for prosthetic control is almost instantaneous since it involves only multiplications and summations. However, the use of MEG for ambulatory prosthetic control is obviously impractical. Preliminary results from our laboratory (F. J. P. Langheim, A. C. Leuthold, J. J. Stanwyck, S. M. Lewis, S. Sponheim, A. P. Georgopoulos, unpublished observations; V. Christopoulos, A. P. Georgopoulos, work in progress) indicate that good motor predictions can be also obtained using EEG signals which are easy to record in an ambulatory setting.

### Example 2

### Prediction Analyses with EEG

We used the same analysis methods for the EEG as those we used for the MEG, which are fully and transparently described in Example 1 above. This method involves two stages.

Stage 1. Obtained suitable X- and Y-coefficients for each EEG channel using linear multivariate regression.

Stage 2. Multiplied (i.e. weight) individual, time-varying EEG signals at time T by the corresponding coefficients and sum across weighted EEG signals to predict the movement trajectory at a future time T+k, where k is typically tens of milliseconds.

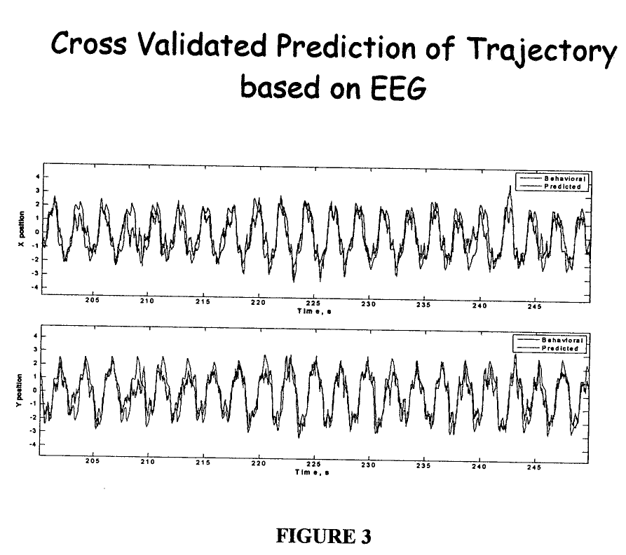

Stage 3. Cross-validation: Used a limited set of the data for Stage 1 analysis to obtain the set of coefficients, and then applied those coefficients to a later set of data for prediction. In this case, the movement data predicted do not contribute to the estimation of the coefficients, hence the cross-validation concept. (This is a standard way in the field of statistical predictions in various fields. See FIG. 3.).

We preprocessed the data to reject artifacts and the “clean” neural signals are then used for movement prediction as above. An example of cross-validated movement prediction using EEG signals preprocessed using independent component analysis is shown in FIG. 3.

With respect to hardware, we have been using a very compact, portable 64-channel (or 32-channel) EEG system manufactured in Germany (Model L64, MKE Medizintechnik für Kinder und Erwachsene GmbH, 56594 Willroth, Germany).

### Example 3

### A Neural Prosthetic

The present invention contemplates using a processed EEG signal from a subject to directly instruct a desired action by the subject, wherein the desired action is an intended movement using the subject's natural limb or a device, such as a prosthetic limb device or a computer screen pointing device. The present invention further contemplates using the knowledge as described in Examples 1-2 above to instruct an intended movement by a human subject, wherein the processed neural signals of intended movement from the human subject are used. The normal neural signaling pathway that directs an intended movement may be rerouted by using the processed neural signal to instruct an intended movement directly to the subject's limb or a device.

### Example 4

### Implementing the Mathematical Models

It is contemplated that algorithms can be implemented using existing commercial hardware (digital and analog). In the future, custom integrated circuits can be designed to reduce power, size and weight; this may allow the entire system to be portable and convenient to the user.

### REFERENCES

Georgopoulos A P, Kettner R E, Schwartz A B (1988) Primate motor cortex and free arm movements to visual targets in three-dimensional space. II. Coding of the direction of movement by a neuronal population. J Neurosci 8:2928-2937
Hinterberger T, Weiskopf N, Veit R, Wilhelm B, Betta E, Birbaumer N (2004) An EEG-driven brain-computer interface combined with functional magnetic resonance imaging (fMRI). IEEE Trans Biomed Eng 51:971-974
Leuthold A C (2003) Subtraction of heart artifact from MEG data: The matched filter revisited. Soc Neurosci Abstr 863.15
McFarland D J, McCane L M, David S V, Wolpaw J R (1997) Spatial filter selection for EEG-based communication. EEG Clin Neurophysiol 103:386-394
Scherer R, Muller G R, Neuper C, Graimann B, Pfurtscheller G (2004) An asynchronously controlled EEG-based virtual keyboard: improvement of the spelling rate. IEEE Trans Biomed Eng 51:979-984
Taylor D M, Tillery S I, Schwartz A B (2002) Direct cortical control of 3D neuroprosthetic devices. Science 296:1829-1832
Vetter R J, Williams J C, Hetke J F, Nunamaker E A, Kipke D R (2004) Chronic neural recording using silicon-substrate microelectrode arrays implanted in cerebral cortex. IEEE Trans Biomed Eng 51:896-904
Wessberg J, Nicolelis M A (2004) Optimizing a linear algorithm for real-time robotic control using chronic cortical ensemble recordings in monkeys. J Cogn Neurosci 16:1022-1035
Wolpaw J R, McFarland D J (2004) Control of a two-dimensional movement signal by a noninvasive brain-computer interface in humans. Proc Natl Acad Sci USA 101:17849-17854
Wolpaw J R, Birbaumer N, Heetderks W J, McFarland D J, Peckham P H, Schalk G, Donchin E, Quatrano L A, Robinson C J, Vaughan T M (2000) Brain-computer interface technology: a review of the first international meeting. IEEE Trans Rehab Eng 8:164-173

What is claimed is:

1. A method for generating a signal indicative of an intended movement which comprises:
a) measuring EEG signals from the brain or nervous system of a moving subject;
b) processing the EEG signals to generate a mathematical model of the intended movement that correlates EEG signals with the intended movement; and
c) obtaining the signal indicative of the intended movement.

2. The method of claim 1, further comprising the step of translating the signal so generated so as to generate a control signal.

3. The method of claim 2, wherein the control signal directs a desired action by the subject.

4. The method of claim 3, wherein the desired action by the subject comprises reaching with a limb.

5. The method of claim 4, wherein the limb is the subject's arm.

6. The method of claim 5, wherein the limb is a prosthetic device attached to the subject.

7. The method of claim 3, wherein the desired action by the subject comprises moving a computer screen pointer device.
